(19) United States
(12) Patent Application Publication
(10) Pub. No.: US 2017/0296795 A1
(43) Pub. Date: Oct. 19, 2017

(54) BALLOON CATHETER VISUALIZATION DEVICE INCLUDING REINFORCEMENT FEATURES

(71) Applicants: BOSTON SCIENTIFIC SCIMED, INC., MAPLE GROVE, MN (US); MAYO FOUNDATION FOR MEDICAL EDUCATION AND RESEARCH, ROCHESTER, MN (US)

(72) Inventors: RACHEL LYNN TROUTMAN, ROCHESTER, MN (US); JAMES M. ANDERSON, CORCORAN, MN (US); JOEL T. EGGERT, PLYMOUTH, MN (US); ROGER W. MCGOWAN, OSTEGO, MN (US); ADAM DAVID GROVENDER, MAPLE GROVE, MN (US); DAVID RAAB, ROSEVILLE, MN (US); BRIAN T. BERG, ST. PAUL, MN (US)

(73) Assignees: BOSTON SCIENTIFIC SCIMED, INC., MAPLE GROVE, MN (US); MAYO FOUNDATION FOR MEDICAL EDUCATION AND RESEARCH, ROCHESTER, MN (US)

(21) Appl. No.: 15/490,487

(22) Filed: Apr. 18, 2017

Related U.S. Application Data
(60) Provisional application No. 62/324,556, filed on Apr. 19, 2016.

(51) Int. Cl. A61M 25/10

(52) U.S. Cl. CPC A61M 25/1002; A61M 2025/1084; A61M 2205/0266

(57) ABSTRACT

A balloon catheter visualization device includes a handle, an elongate shaft, and an asymmetrical balloon. The elongate shaft includes a distal end, a proximal end, and defines a lumen and a longitudinal axis extending from the proximal end to the distal end. The proximal end is coupled to the handle. The asymmetrical-shaped balloon including a distal portion and a proximal portion. The proximal portion is coupled to the distal end of the elongate shaft. The proximal portion has one or more reinforcement features including a layer of material coupled to the asymmetrical-shaped balloon and forming an outer contour at the proximal portion of the asymmetrical-shaped balloon that is asymmetrical to an outer contour of the distal portion of the asymmetrical-shaped balloon.

### CROSS-REFERENCE TO RELATED APPLICATIONS

This application claims priority under 35 U.S.C. §119(e) to U.S. Provisional Application No. 62/324,556, filed Apr. 19, 2016, the entire disclosure of which is herein incorporated by reference.

### TECHNICAL FIELD

This document relates to medical devices, such as, for example, balloon catheter visualization devices.

### BACKGROUND

The presence and movement of opaque bodily fluids such as blood generally make in vivo imaging of tissue regions within a patient difficult. Medical devices may therefore, in some cases, be used to visualize interior regions of a patient's body by depicting a visual construct. For example, ultrasound devices may be used to produce in vivo ultrasound images from within a body. In another example, mapping devices having position sensors for generating a map depicting a two- or three-dimensional image of a patient's interior region may also be used. Visual information provided by such devices can be limited in some cases. Mapping devices, for example, may not be able to provide visual information of the tissue surface condition within a heart chamber. Thus, there is a need for medical devices that provide improved visualization for viewing a blood-filled cavity or vessel within the patient.

Medical devices may, in some cases, use an inflatable imaging balloon to obtain in vivo imaging of the patient's tissue regions. The imaging balloon can be introduced into the patient's body in a deflated state. Once introduced, the imaging balloon can be inflated and pressed against a targeted tissue region for imaging. Imaging can be achieved by use of an optical fiber or other electronic apparatus for viewing tissue through the wall of the inflated balloon.

Imaging balloons may often encounter issues that affect the quality of the image being received. Imaging balloons may produce poor or blurred tissue images if the balloon is not firmly pressed against the tissue surface.

### SUMMARY

This document relates to medical devices, such as, for example, balloon catheter visualization devices. In particular, this document relates to a reinforced balloon of a catheter visualization device or system for visualizing an anatomy within a patient's vasculature during a diagnostic or surgical medical procedure.

In some aspects, a balloon catheter visualization device includes a handle, an elongate shaft, and an asymmetrical-shaped balloon. The elongate shaft can include a distal end and a proximal end. The proximal end can be coupled to the handle. The elongate shaft can define a lumen and a longitudinal axis, both extending from the proximal end to the distal end of the shaft. The asymmetrical-shaped balloon includes a distal portion and a proximal portion. The proximal portion is coupled to the distal end of the elongate shaft.

The proximal portion of the asymmetrical-shaped balloon contains one or more reinforcement features including a layer of material coupled to the asymmetrical-shaped balloon and forming an outer contour at the proximal portion of the balloon that is asymmetrical to an outer contour of the distal portion of the asymmetrical-shaped balloon.

In some cases, the asymmetrical-shaped balloon includes multiple reinforcement features that form a plurality of ribs. In some cases, the plurality of ribs are located circumferentially equidistant from one another. In some cases, the one or more reinforcement features include at least one diametrically expandable rib. The at least one diametrically expandable rib can define a lumen sized to receive an inflation media for diametrically expanding the rib. The at least one diametrically expandable rib can be configured to inflate independently of the asymmetrical-shaped balloon. In some cases, the asymmetrical-shaped balloon catheter visualization device includes up to six or more reinforcement features.

In some cases, the balloon catheter visualization device can include one reinforcement feature comprising the layer of material uniformly disposed over the proximal portion of the asymmetrical-shaped balloon. The proximal portion can have an average wall thickness that is greater than an average wall thickness of the distal portion. In some cases, a thickness ratio of a balloon wall coupled to the reinforcement feature in comparison to the balloon wall alone is about 1:1, about 2:1, about 3:1, about 4:1, about 5:1, or greater than 5:1. In various cases, the one or more reinforcement features are configured to prevent balloon material folding at the proximal portion of the asymmetrical-shaped balloon, when the distal portion of the asymmetrical-shaped balloon is subjected to compressional force.

In some aspects, a balloon catheter visualization device includes a handle, an elongate shaft, a balloon, and a reinforcement feature. The elongate shaft includes a distal end and a proximal end. The proximal end can be coupled to the handle. The elongate shaft defines a lumen and a longitudinal axis that extend from the proximal end to the distal end. The balloon includes a proximal end coupled to the distal end of the elongate shaft, a distal portion, and a wall defining an inner inflation cavity. The reinforcement feature is disposed within the inner inflation cavity and includes a plurality of flexible struts. Each struts is connected to at least two adjacent struts. The reinforcement feature is configured to include a collapsed configuration for providing a small profile for suitable for delivery and an expanded configuration for providing structural support to the balloon.

In some cases, the reinforcement feature in the expanded configuration can form a frame having a flared cone shape. The reinforcement feature can be a separate component configured to be inserted into the balloon and expanded therein. The reinforcement feature can be coupled at one or more locations of the balloon. The reinforcement feature can optionally be coupled to a polymeric covering. In some cases, the reinforcement feature includes a plurality of interconnected struts, each individual strut forming a petal-like shape when the reinforcement feature is in the expanded configuration. The reinforcement feature can be made of nitinol, in some cases. In some cases, the plurality of flexible struts are interconnected such that the struts collapse and expand together.

In some aspects, a balloon catheter visualization device includes a handle, an elongate shaft, and a balloon. The elongate shaft includes a distal end and a proximal end. The proximal end can be coupled to the handle. The elongate shaft defines a lumen and a longitudinal axis that extend from the proximal end to the distal end. The balloon includes a proximal end and a distal end. The proximal end of the balloon can be coupled to the elongate shaft and include a means for reinforcing the balloon to prevent material folding at the proximal end of the balloon when force is applied to the distal end of the balloon.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is an illustration of an exemplary balloon catheter visualization device system within a human anatomy.

FIG. 2 is a perspective view of a distal end portion of the exemplary balloon catheter visualization device of FIG. 1.

FIGS. 3A and 3B are a side view and a cross-sectional view of a distal end of a second example of a visualization balloon catheter.

FIG. 4 is a side view of third example of a balloon catheter visualization device provided herein.

FIGS. 5A and 5B shows the visualization balloon catheter of FIG. 3 during force testing.

FIG. 6 shows the visualization balloon catheter of FIG. 4 during force testing.

FIG. 7 provides force versus compression length data of the visualization balloon catheter of FIG. 1 in comparison to a control balloon catheter.

FIGS. 8A-8C are a side view and cross-sectional views of a fourth example of a balloon catheter visualization device provided herein.

FIG. 9 is a side view of a fifth example of a balloon catheter visualization device provided herein including a reinforcement structure.

FIGS. 10A-10C are side, plan, and perspective views of another example of a reinforcement structure provided herein.

FIG. 11 is a side view of a sixth example of a balloon catheter visualization device provided herein.

FIG. 12 is a side view of a seventh example of a balloon catheter visualization device provided herein.

### DETAILED DESCRIPTION

Catheter visualization devices provided herein can be used for various medical purposes. For example, in some cases, weeping balloons provided herein are used for catheter visualization devices and systems. In some cases, the weeping balloons, as described herein, can include reinforcement features (also referred to reinforcement structures or frames) that provide the balloons with increased mechanical integrity and strength (e.g., stiffness). In some cases, the reinforcement features can be coupled to the balloon material, for example, bonded to the balloon wall or encapsulated within the balloon wall. In some cases, the reinforcement features provided herein are additional components or devices that can be used in conjunction with the balloon (e.g., components or devices insertable within a lumen or inner cavity of the balloon). As described further below, the design of the reinforcement features can affect performance characteristics such as, but not limited to, resistance to buckling, bending, and balloon material folding during use.

Balloon catheter visualization device, systems, and methods provided herein can allow for balloon catheter visualization of a target location, which can provide anatomy and pathology identification as well as device placement visual feedback to the physician user during a minimally invasive method. Balloon catheter visualization devices, systems, and methods provided herein can include an elongate, compliant balloon having a transparent wall. In some cases, the balloon wall (e.g., a transparent balloon wall) can have one or more reinforcement features that limits the buckling, bending, and material folding of the balloon during use. In some cases, the reinforcement features can include metallic or polymeric materials that are bonded to or embedded within the balloon wall. In some cases, the reinforcement features can include a separate component or device configured to be insertable within the balloon.

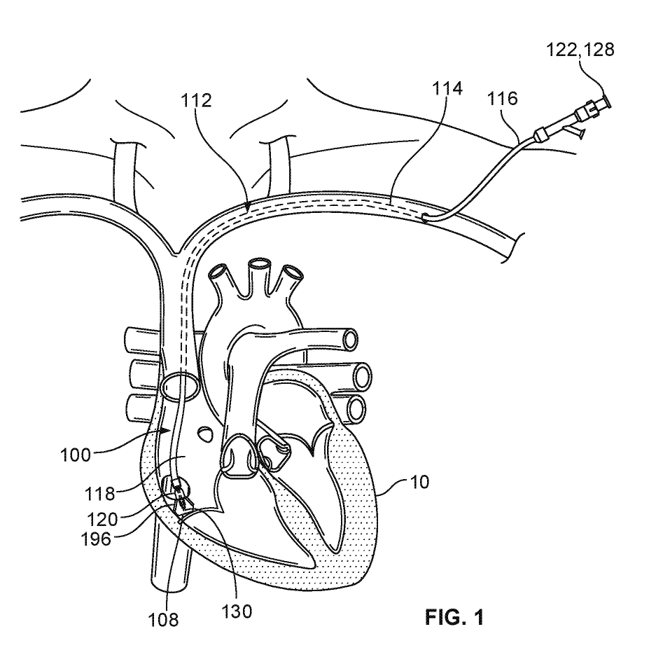

Referring to FIG. 1, an exemplary balloon catheter visualization system 100 can be used to visualize tissue structures within a human anatomy. In some cases, the balloon catheter visualization system 100 can be inserted into a right atrium of a heart 10 through a brachial vein or a jugular vein. The balloon catheter visualization system 100 includes a tubular body 112 (which can also be described as an elongate shaft or catheter) having a proximal end portion 114 with a proximal end 116 and a distal end portion 118 with a distal end 120. In some cases, proximal end portion 114 can couple to a catheter hub 122 or a manifold. In the depicted embodiment, the distal end portion 118 includes an integrated camera (not shown), and at least one balloon 108 (also described as balloon member).

As shown in FIG. 1, the balloon 108 can form a distal tip of balloon catheter visualization system 100. In some cases, the balloon 108 can encapsulate the distal end 120 of the distal end portion 118 such that the integrated camera is disposed within the balloon 108. In some cases, the balloon 108 includes a thru lumen 130 that extends from one end of the balloon 108 to an opposite end of the balloon 108. The thru lumen 130 can be sized to receive ancillary components and devices (not shown), such as a fastening tool or a surgical device. The ancillary components and devices can be passed through the thru lumen 130 of the balloon 108 for accessing an anatomical location outside the balloon 108.

In some cases, balloon 108 is a weeping balloon. More specifically, in some cases, the balloon can include pores to allow for the balloon to “weep” to provide a visually clear area surrounding the balloon. The pores of the balloon 108 can be configured to slowly transmit fluid through a wall of the balloon 108. Such fluid can be visually clear or transparent and can displace blood that would otherwise obscure visualization of areas adjacent to the balloon 108.

Certain embodiments of the balloon 108 include a reinforced balloon. In particular, the balloon can include one or more reinforcement features 196. The reinforcement features 196 can provide the balloons with increased mechanical integrity and strength (e.g., stiffness). In some cases, as shown in FIG. 1, the reinforcement features 196 can be coupled to the balloon material. The reinforcement features 196 may be adhesively or heat bonded directly to the balloon wall. In some cases, the reinforcement features 196 can be encapsulated within the balloon wall. In some cases, the reinforcement features 196 are additional components or devices that can be used in conjunction with the balloon, e.g., components or devices insertable within a lumen or inner cavity of the balloon, as discussed herein.

In some cases, balloon catheter visualization system 100 includes at least one tubular body 112 defining a lumen (not shown). In some cases, balloon catheter visualization system 100 can include multiple tubular bodies, in which each tubular body defines at least one lumen. Each tubular body 112 can optionally include multiple lumens, for example, coaxial or non-coaxial lumens. Balloon catheter visualization system 100 can have one or more lumens that extend partially or fully thorough one or more tubular bodies 112. One or more lumens can be used as a conduit adapted to receive components (e.g., integrated camera or fastener tools, and/or inflation media, e.g., saline). In some cases, one or more lumens can be adapted to jet inflation media (e.g., saline), into distal end portion 118 of balloon catheter visualization device 100.

In some cases, catheter hub 122 generally connects an external fluid supply to one or more lumens of balloon catheter visualization system 100. Catheter hub 122 can include one or more ports 128 to facilitate a fluid connection to another medical device or a fluid source. For example, port 128 can supply saline solution into one or more lumens of tubular body 112. Catheter hub 122 may be coupled to tubular body 112 directly or indirectly. In some cases, a flexible tubing, sometimes referred to as a strain relief tubing, is coupled between hub 122 and the tubular body 112 at the proximal end 116 to provide a longitudinal tapered transition between catheter hub 122 and tubular body 112. Flexible tubing can help to increase kink resistance of tubular body 112 at proximal end portion 114.

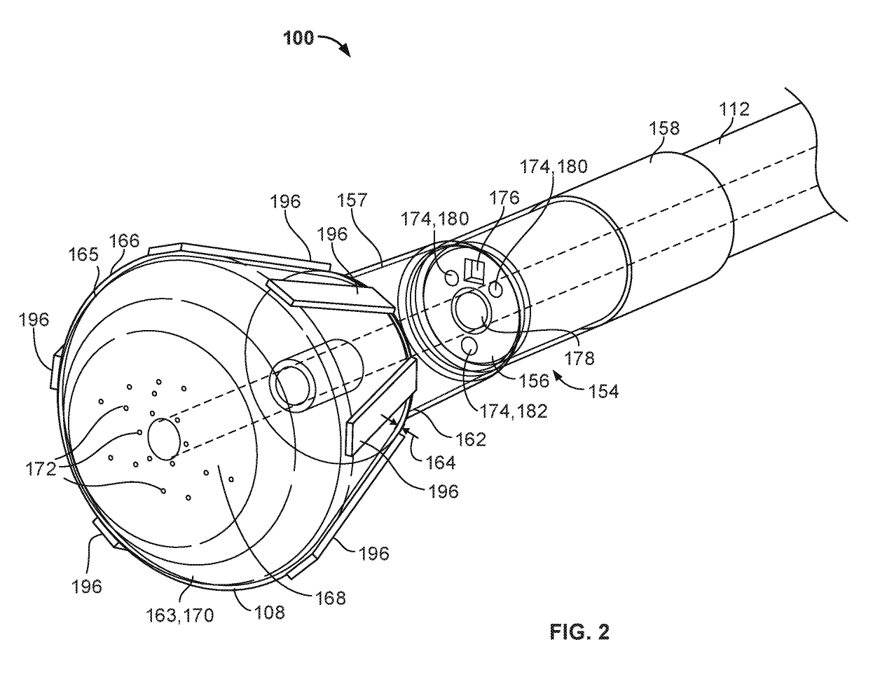

Referring to FIG. 2, a distal end of exemplary balloon catheter visualization system 100 can include the balloon 108. The balloon catheter visualization system 100 can include an elongate, tubular body 112 with a distal end portion 154. A distal end 156 of distal end portion 154 can be either directly or indirectly coupled to a balloon 108. For example, tubular body 112 can be coupled to balloon 108 indirectly by using an optional intermediate catheter shaft 157. The intermediate catheter shaft 157 can couple to a proximal end 162 of balloon 108 and a catheter interface portion 158 of tubular body 112.

In some cases, balloon 108 is disposed at or near to distal end 156 of tubular body 112. Balloon 108 can include the proximal end 162, a distal end 163, and a wall 164 that extends from an interior surface 165 to an exterior surface 166. In the depicted embodiment, balloon 108 forms a distal tip 170 of balloon catheter visualization system 100. As described further herein, balloon 108 can be filled with an inflation media in an interior cavity 168 defined by the walls of the balloon. Balloon 108 can be a weeping balloon device (e.g., a balloon structure that defines one or more openings or perforations 172 extending through wall 164). Balloon 108 can have a distal face that defines the perforations 172 of the weeping balloon 108. In such a case, the distal face of the balloon 108 can be abutted to tissue and the tissue can be visualized using the balloon catheter visualization system 100.

Still referring to FIG. 2, in some cases, the distal end of tubular body 112 can include a plurality of lumens 174. Each lumen of plurality of lumens 174 can longitudinally extend within tubular body 112 (entirely or partially therethrough). Each lumen 174 can be formed from one of various cross-sectional shapes (e.g., circle, oval, slot, square, rectangular, triangular, trapezoid, rhomboid, or irregular shape). The shape of the lumen may facilitate receiving other components of balloon catheter visualization system 100. For example, one or more lumens 174 can be used to receive a fastening tool (not shown), a camera 176, fiber optic light cables (not shown), electrical cables (not shown), inflation media and combinations thereof. In some cases, tubular body 112 defines a central lumen 178 for receiving a fastening tool (not shown) for delivering a fastener (not shown), two lumens for receiving fiber optic light cables 180, one lumen for delivering inflation media 182, and one lumen for receiving camera 176.

In some cases, balloon 108 of balloon catheter visualization system 100 is a weeping balloon, which is a balloon structure defining one or more perforations 172 (also described as apertures, hole, slits, openings, pores, micropores, etc., extending through a balloon wall) through which fluid can pass. As such, weeping balloons 108 can transfer fluid through the balloon wall 164, from the interior cavity 168 to the exterior surface of balloon 108. Transferring fluid (e.g., inflation media) to the exterior surface 166 can provide a benefit of displacing blood from exterior surface 166 of balloon 108 that would otherwise blur or obstruct visual imaging through balloon 108. In other words, inflation media transferred through the one or more perforations 172 can help keep the exterior surface 166 of balloon 108 visually clear. When a plain balloon is placed against an anatomical surface, blood can be trapped on the balloon surface and thus obscure the view, but inflation media (e.g., saline) exiting the perforations 172 of the weeping balloon 108 can wash away this blood on the balloon surface adjacent to the anatomical surface.

Still referring to FIG. 2, the weeping balloon 108 used in a balloon catheter visualization system 100 or other medical device can include one or more reinforcement features 196 (which can also be referred to as reinforcement structures) configured to provide the balloon 108 with structural support during a medical procedure. In some cases, the balloon 108 can include multiple reinforcement features 196 (e.g., two, three, four, five, six, seven, eight, ten, twenty, thirty, or forty features). Each reinforcement feature 196 (which can also be called a rib, or a strip) can be formed into an elongate piece coupled to the exterior surface 166 of the balloon 108. The reinforcement features 196 can be located at any one or more locations on the balloon 108 to provide additional strength or stiffness in select areas. For example, one or more reinforcement features 196 can be placed along a proximal (or distal) portion of the balloon 108. The reinforcement features 196 may be oriented such that each feature 196 extends along the balloon 108 in a particular direction (e.g., each feature 196 may extend distally along the body of the balloon 108). In some cases, the reinforcement features 196 may be positioned circumferentially equal-distant from one another on the balloon 108 to minimize biased bending in a given direction.

In some cases, the reinforcement features 196 can provide the balloon 108 with added column strength and structural support at one or multiple desired locations along the balloon body. The reinforcement features 196 can help keep the balloon 108 aligned with the intermediate catheter shaft 157 when the distal end of the system 100 is pushed against a patient's anatomy (e.g., tissue). In some cases, the reinforcement features 196 can prevent or reduce bending or bucking of the balloon 108 at or near its proximal end (or other desired location) by providing added stiffness to the select portions of the balloon 108. In some cases, the reinforcement feature 196 can prevent the wall 164 of the balloon 108 from folding over on itself (see FIG. 6) when the balloon 108 is pushed against tissue. Accordingly, the balloon 108 with reinforcement feature design embodiments provided herein can provide improved performance in comparison to prior conventional balloon devices.

Certain embodiments of the reinforced balloon 108 define the perforations extending through both the balloon 108 and the one or more reinforcement features 196. The reinforcement features 196 can supplement the stiffness of balloon wall 166 around the perforations 172 to provide greater resistance to the deformation of the perforations 172 that might otherwise occur without the presence of the reinforcement features 196. For example, as the fluid pressure within the internal space defined by the balloon 108 increases, the balloon wall 164 will tend to stretch. The stretching of the balloon wall 164 will tend to enlarge the perforations 172. For example, as the perforations 172 enlarges, the perforations 172 may become too large and, in result, more fluid than may be desired is transmitted through the perforations 172.

While a visualization system 100 is used to describe the reinforced balloons 108 provided herein, it should be understood that the visualization system 100 is just one example implementation. The reinforced balloon devices provided herein can be used in various other implementations. For example, in some cases, the reinforced balloon devices provided herein can be used to deliver a therapeutic agent or a device while providing visual feedback to the user.

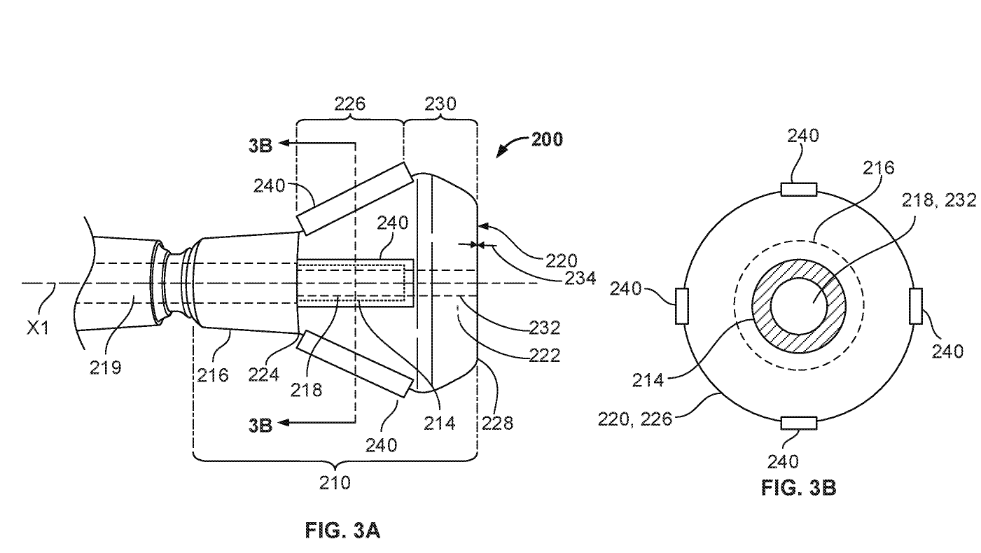

Referring to FIGS. 3A and 3B, another example of a visualization balloon system 200 includes an optional inner member 214, an outer shaft 216, and a weeping balloon 220. The outer shaft 216 couples to a proximal end 224 of the balloon 220, which is located at a distal end 210 of the system 200. The outer shaft 216 can be coupled to a manifold at a proximal end of the system. The outer shaft 216 can define one or more lumens 219 for receiving other components or devices. For example, the lumen 219 may be sized for receiving a portion of the inner member 214 and/or other components (e.g., electrical and imaging connectors coupled to an imaging element). The outer shaft 216 can optionally be configured to receive other additional components, such as a reinforcement frame (not shown) configured to deploy within a balloon inner cavity, which will be discussed in later sections.

In some cases, the inner member 214 can be coupled to the balloon, the distal end of the outer shaft, or both. As shown in FIGS. 3A and 3B, the inner member 214 defines a lumen 218 at the proximal portion 226 of the balloon 220. The lumen 218 of the inner member 214 can be communicatively coupled with a thru lumen 232 formed by the balloon wall at the distal portion 230 of the balloon 220. The inner member 214 can reinforce material that forms the thru lumen by, for example, providing a layer of material to portions of the balloon wall forming the thru lumen 232 of the balloon 220. Such reinforcement provided by the inner member can allow components to be passed through the lumens 218, 232 without damaging the balloon 220. In some cases, the inner member 214 can define multiple lumens extending therethrough. In some cases, the inner member 214 can include a lumen 218 sized for receiving the imaging element and electrical leads and imaging connectors coupled to the imaging element. In some cases, the inner member 214 can include a lumen 218 sized for receiving other medical devices.

In some cases, an imaging element (not shown) may be coupled to the inner member 214, or the outer shaft 216 to receive visual images of tissue that comes in contact with the balloon 220 and send the visual information to a receiver connected to the proximal end of the system 200. The imaging element can include a variety of optical receivers, such as a camera.

Still referring to FIGS. 3A and 3B, the balloon 220 can be coupled to the outer shaft 216 to allow the visualization balloon system 200 to receive optical imaging of tissue within a blood-filled vessel or cavity. In particular, the balloon 220 can be used to directly contact tissue within a blood-filled vessel or cavity such that the imaging element can capture optical imaging of the contacted tissue. As shown, the depicted balloon 220 includes the proximal end 224, a proximal portion 226, a distal end 228, and a distal portion 230. A wall 234 (and optionally also the thru lumen 232) of the balloon 220 can define an inner cavity 222 configured to receive an inflation medium (e.g., saline solution) for inflating the balloon 220. The inner cavity 222 of balloon 220, when inflated, provides a visible region within the balloon 220 that allows the imaging element to visualize tissue that comes in contact with the balloon wall 234.

Various embodiments of the balloon 220 provided herein can have a range of suitable dimensions for providing adequate structural integrity to withstand a desired inflation pressure during a medical procedure. For example, when the balloon 220 is in a collapsed state, the balloon 220 can be folded into a reduced profile having a suitable maximum diameter. In some cases, a collapsed, folded balloon 220 can have a reduced profile with a maximum diameter ranging from about 3 mm to about 5 mm (e.g., from about 3 mm to about 3.5 mm, from about 3.5 mm to about 4 mm, or from about 4.5 mm to about 5.0 mm). When the balloon 220 is an expanded state, the balloon 220 can be inflated to provide a visual pathway through the inner cavity 222 of the balloon 220. The balloon 220 can expand to a suitable maximum diameter when pressurized with an inflation medium (e.g., saline solution). In some cases, the balloon 220 can inflate to a maximum diameter ranging from about 5 mm to about 20 mm (e.g., from about 5 mm to about 7 mm, from about 7 mm to about 10 mm, from about 10 mm to about 15 mm, or from about 15 mm to about 20 mm).

Various embodiments of the balloon 220 can include one or more reinforcement features 240, where each reinforcement feature 240 is disposed on an exterior surface of the balloon wall 234 at one or more locations, for example, locations at the proximal portion 226 of the balloon 220. The reinforcement features 240 may be oriented such that an elongate portion of each feature 240 extends in the same direction as the longitudinal axis “X1” of the balloon 220. The alignment and location of the reinforcement feature 240 can affect the balloon's resistance to bending and buckling when the balloon 220 is subjected to a compression force. As such, in some cases, the reinforcement features 240 may provide the balloon 220 with added column strength or stiffness.

Reinforcement features 240 can be coupled at one or more location of the balloon 220. As shown in FIGS. 3A and 3B, the reinforcement features may be coupled to the exterior wall of the balloon 220. In some cases, the reinforcement feature 240 can be disposed on an interior wall of the balloon 220. In some cases, the reinforcement feature 240 can be disposed at least partially within the wall of the balloon 220.

In some cases, an optional layer of material (not shown) can be disposed over the reinforcement feature 240 to secure the reinforcement features 240 to the balloon 220. The reinforcement feature 240 can be encapsulated by the layer of material (e.g., PEBAX®) when sandwiched between the balloon wall 234 and the layer of material. The layer of material may be disposed over at least a portion of each reinforcement feature 240, over the entire portion of each reinforcement feature 240, or over the entire balloon 220.

In some cases, the balloon 220 includes a hydrophilic coating. The hydrophilic coating can be disposed over at least a portion of an exterior surface of the balloon wall 234. For example, the hydrophilic coating can be disposed over the exterior surface of the proximal portion 226 of the balloon 220, or the entire exterior surface of the balloon 220. In some cases, the balloon 220 incorporates material containing a hydrophilic polymer. For example, the hydrophilic polymer can be incorporated into the balloon material (e.g., the balloon can be formed using a hydrophilic polymer blend). Exemplary hydrophilic polymers and coatings can include, but are not limited to, a polyvinylpyrrolidone and a polyurethane.

The balloon 220 provided herein can include various different shapes. For example, in some cases, the balloon 220 can form a tear shape, a spherical shape, a donut shape, an ovoid shape, or any irregular shape. The reinforcement features 240 can affect the shape of the balloon 220 depending on its forms (e.g., strips, ribs, or a layer) and its location on the balloon 220. As such, the balloon 220 may include various asymmetrical shapes (e.g., contours) that can be defined by the balloon wall and the reinforcement features 240. In some cases, the asymmetrical balloon 220 has a surface contour along its proximal portion 226 that is different from a surface contour along its distal portion 230. For example, as shown in FIG. 3, the exterior surface of the proximal portion 226 of the balloon 220 includes ridge-like protrusions formed by the reinforcement features 240. In contrast, the distal portion 230 of the balloon 220 has no attached reinforcement features 240, and therefore has a smooth rounded surface having no protrusions. In some cases, the distal end 228 of the balloon 220 is formed into a flat distal face configured for seating the balloon 220 flush against a planar surface.

The balloon 220 can be made of a polymer, such as an elastomeric material (e.g., silicone), to allow the balloon material to expand when inflated, and the reinforcement features can be made of a metal (e.g., stainless steel or nitinol) or a polymer (e.g., a polyether block amide (e.g., PEBAX®), a polyurethane, or a silicone). In some cases, the balloon 220 and the reinforcement feature 240 are made of the same material. In some cases, the balloon 220 and the reinforcement feature 240 are made of different materials. For example, in some cases, the reinforcement feature 240 is made of polyether block amide (e.g., PEBAX®) while the balloon wall is made of silicone (e.g., about 30A durometer). It should be understood that other combinations of materials are also envisioned. In another non-limiting example, in some cases, the balloon wall is made of silicone at a first durometer (e.g., about 30A durometer) and the reinforcement feature 240 is made of silicone at a second durometer (e.g., about 50A durometer).

The reinforcement features 240 can include various suitable shapes and dimensions configured for provided added support to portions of the balloon 220. Non-limiting examples of possible shapes of the reinforcement feature 240 include strips, rods, tubes, braids, and layers of material. For example, the reinforcement features 240 can include one or more strips of a material having a suitable length, width, and thickness. In some cases, the reinforcement feature 240 has a length for extending the reinforcement feature along the entire or portions of the proximal portion of the balloon 220. In some cases, the reinforcement feature 240 has a length that extends over a portion of or the entire balloon 220 (e.g., from its proximal end 224 to its distal end 228). Suitable lengths of the reinforcement feature 240 can range from about 2 mm to about 20 mm (e.g., from about 5 mm to about 15 mm, from about 8 mm to about 12 mm, from about 15 mm to about 20 mm, from about 3 mm to about 10 mm, from about 2 mm to about 5 mm, from about 2 mm to about 3 mm, from about 2 mm to about 4 mm, from about 5 mm to about 10 mm, or from about 5 mm to about 7 mm).

The reinforcement feature 240 can include a width and thickness for providing suitable column strength to minimize bending, buckling, or folding of the balloon 220. In some cases, the reinforcement feature 240 has a width of about 0.5 mm to about 2 mm (e.g., from about 0.5 mm to about 1.0 mm, from about 1.0 mm to about 1.5 mm, or from about 1.5 mm to about 2 mm). In some cases, the reinforcement feature 240 has a thickness of about 0.02 mm to about 2 mm (e.g., from about 0.025 mm to about 1 mm, from about 0.03 mm to about 0.5 mm, from about 0.05 mm to about 0.20 mm, from about 0.25 mm to about 0.5 mm, from about 0.5 mm to about 1.0 mm, from about 1.0 mm to about 1.5 mm, or from about 1.5 mm to about 2 mm).

In some cases, the reinforcement feature 240 can be attached to the balloon wall. In some cases, an adhesive is used to bond the reinforcement feature 240 to the balloon wall. For example, in some cases a UV curable silicone adhesive is used to bond the reinforcement feature 240 to the balloon wall. Other types of adhesives can also be used. In some cases, the reinforcement feature 240 is molded onto the balloon wall.

Still referring to FIGS. 3A and 3B, the balloon can include a lumen 232 (and optionally an inner member with a lumen 216) having a suitable inner diameter for allowing passage of components or other devices through the balloon 220. The inner diameter of the thru lumen 232 can range from about 0.3 mm to about 3 mm (e.g., from about 0.4 mm to about 2 mm, from about 0.5 mm to about 1 mm, from about 1 mm to about 1.5 mm, from about 1.5 mm to about 2.0 mm, from about 2.0 mm to about 2.5 mm, or from about 2.5 mm to about 3.0 mm).

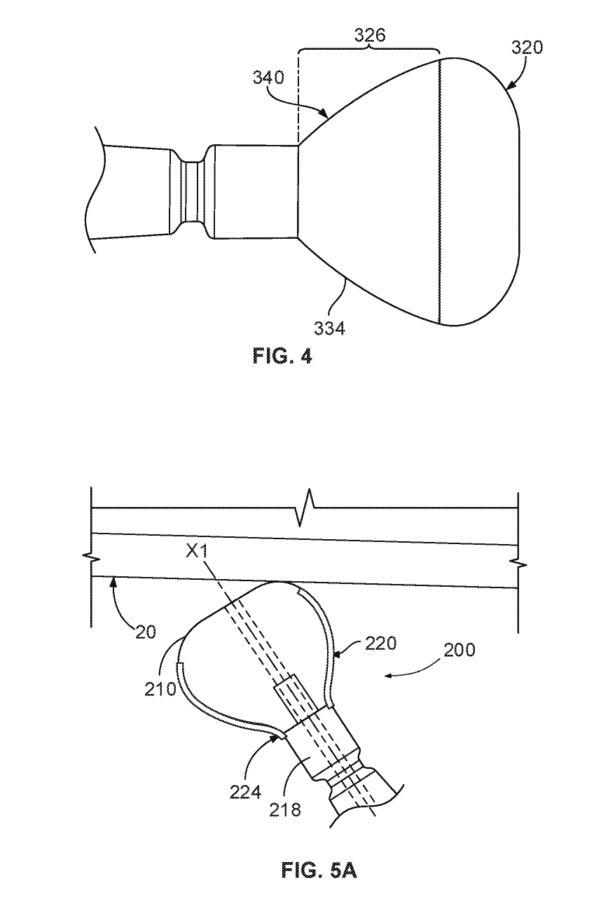

Referring to FIG. 4, another example of a weeping balloon 320 includes a reinforcement feature 340 in the form of a polymer layer disposed over a proximal portion 326 of the balloon 320. In some cases, the reinforcement feature 340 can include a polymer layer disposed over any portion of the balloon 320 suitable for strengthening the covered portions of the balloon. For example, the reinforcement feature 340 can include the polymer layer disposed over a portion of, or an entire proximal portion 326 of the balloon 320. In some cases, the reinforcement feature 340 can be in the form of a polymer layer disposed over multiple regions of the balloon 320 (e.g., the distal and proximal ends but not the midsection of the balloon 320).

The reinforcement feature 340 can be designed to allow the balloon to remain aligned with the outer shaft during use. For example, in some cases, the reinforcement feature 340 includes reinforcing elements (not shown) embedded within the polymer layer, such as fibers, nanotubes, fillers such as composite or metallic particles, to increase the mechanical properties (e.g., stiffness) of the polymer layer. In some cases, the reinforcing elements can be aligned in a particular direction within the polymer layer to promote anisotropic strength or stiffness to the balloon 320.

In some cases, the reinforced portions of the balloon 320 can provide the balloon 320 with an increased wall thickness in select areas. For example, the reinforcement feature 340 shown in FIG. 4 adds a layer of material to the proximal portion 326 of the balloon wall 334. The layer of material can be uniformly disposed over the proximal portion of the balloon 320. In some cases, the proximal portion has an average wall thickness that is greater than an average wall thickness of the distal portion. In some cases, a thickness ratio of a balloon wall 334 coupled to the reinforcement feature 340 in comparison to the balloon wall 334 alone is about 1:1, about 2:1, about 3:1, about 4:1, about 5:1, or greater than 5:1.

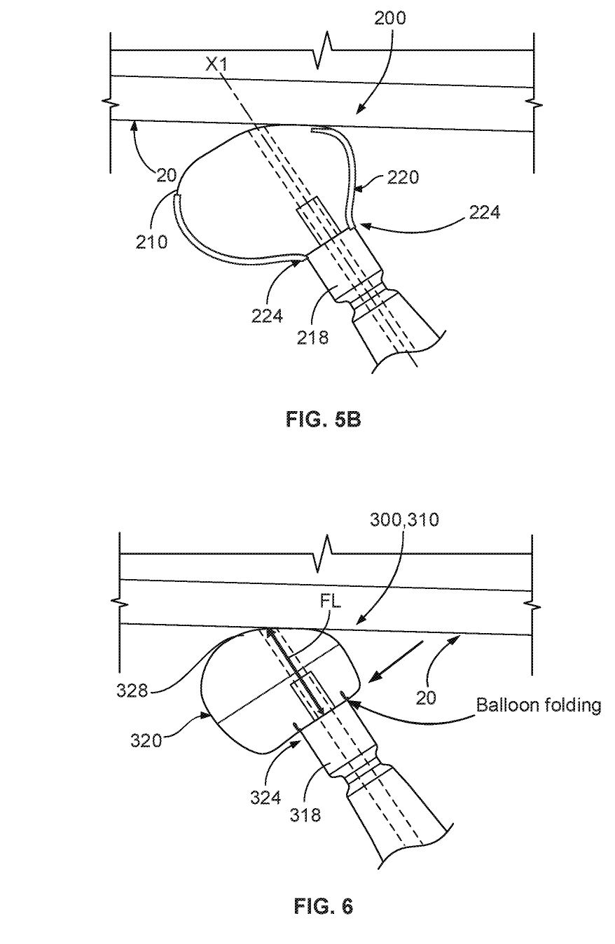

Referring to FIGS. 5A and 5B, the visualization balloon system 200 of FIG. 3 is shown with its distal end 210 positioned against a surface 20 at an angle (approximately an angle of 45°) relative to the longitudinal axis “X1” of the outer shaft 216. In FIG. 5A, the balloon 220 is shown contacting the surface 20, but minimal or no force is being applied to the balloon 220. In FIG. 5B, the balloon 220 is shown being pushed against the surface 20. As shown, the applied force compressed a portion of the balloon 220 away from the surface 20, but the balloon remained generally aligned with the outer shaft 216 of the system 200 (e.g., did not buckle). The balloon 220 shown in FIG. 5B did not buckle at its proximal end 224, where the balloon 220 couples to the shaft 216. Furthermore, the wall of the balloon 220 of FIG. 5B did not fold over on itself.

Referring to FIG. 6, another example of a visualization balloon catheter system 300 includes the balloon 320 of FIG. 4 pushed against a surface 20 with the system 300 positioned at an angle (e.g., about 45°) relative to the surface. The applied force caused the balloon wall at a proximal end 324 of the balloon 320 to fold over on itself. Furthermore, the folding of the balloon 320 resulted in the overall length of the balloon 320 to decrease, changing the focal length “FL” between an imaging element within the balloon 320 and the distal tip 328 of the balloon 320. It can be noted that the applied force compressed a portion of the balloon, however, did not change the general alignment of the balloon 320 relative to outer shaft 318 (e.g., did not buckle). Although the balloon 320 of FIG. 4 exhibited a small degree of balloon folding, as shown in FIG. 6, the balloon 320 did not buckle or bend at the proximal end 324 of the balloon 320. It may be contemplated by a skilled artisan that further modifying (e.g., increasing) the wall thickness of the reinforcement feature 340 (e.g., polymer layer) at the proximal portion of the balloon 320 may minimize or prevent balloon material folding.

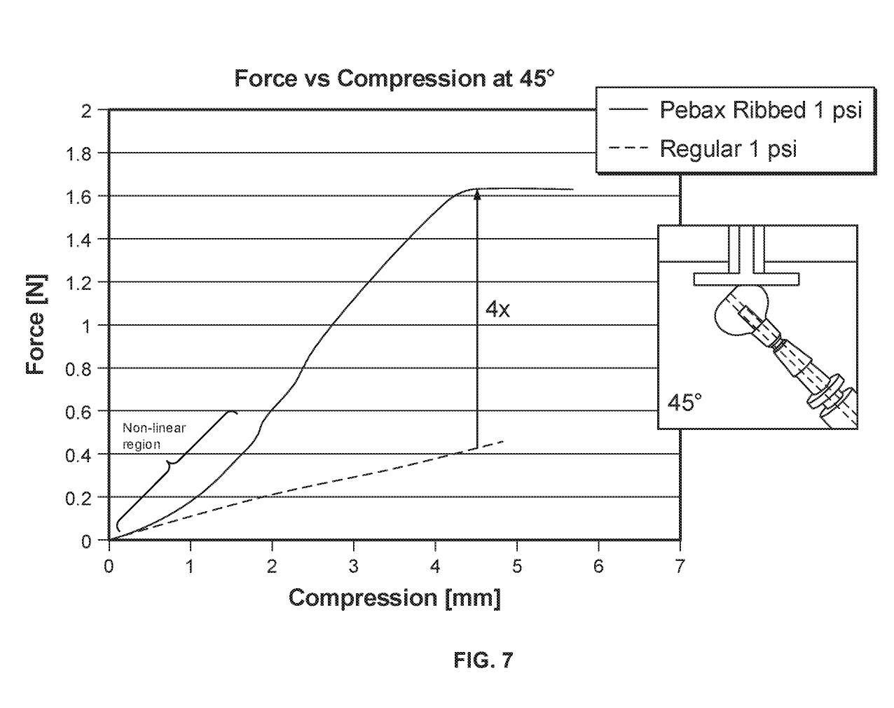

Referring to FIG. 7, a graph provides force data versus compression length data of the visualization balloon system of FIG. 3 (“test sample”) and a system having a weeping balloon with no reinforcement features (“control sample”), as collected from an Instron Testing Equipment. Each sample was tested by applying angular compression. Angular compression was applied to each sample by lowering a flat plate against each device positioned at a 45° angle relative to the plate. As demonstrated by FIG. 7, the test sample showed initially a non-linear relationship between the applied force and the compression length. In contrast, the control sample exhibited a consistent linear relationship between the applied force and the compression length. When comparing the test sample to the control sample, a significantly higher force was generally required to compress the test sample. The difference in force between the test sample and the control sample steadily increased when the samples were compressed between 1 mm to 4 mm. The results showed that the test sample required a higher application of angular compressional forces than the control sample for compressing the balloon. In particular, the reinforcement features appear to provide the balloon of the test sample with four times the stiffness at its peak compression force reading when compared to the control sample at the same internal pressure. Accordingly, the reinforcement features were demonstrated to provide additional structural support to the test sample as compared to the control sample.

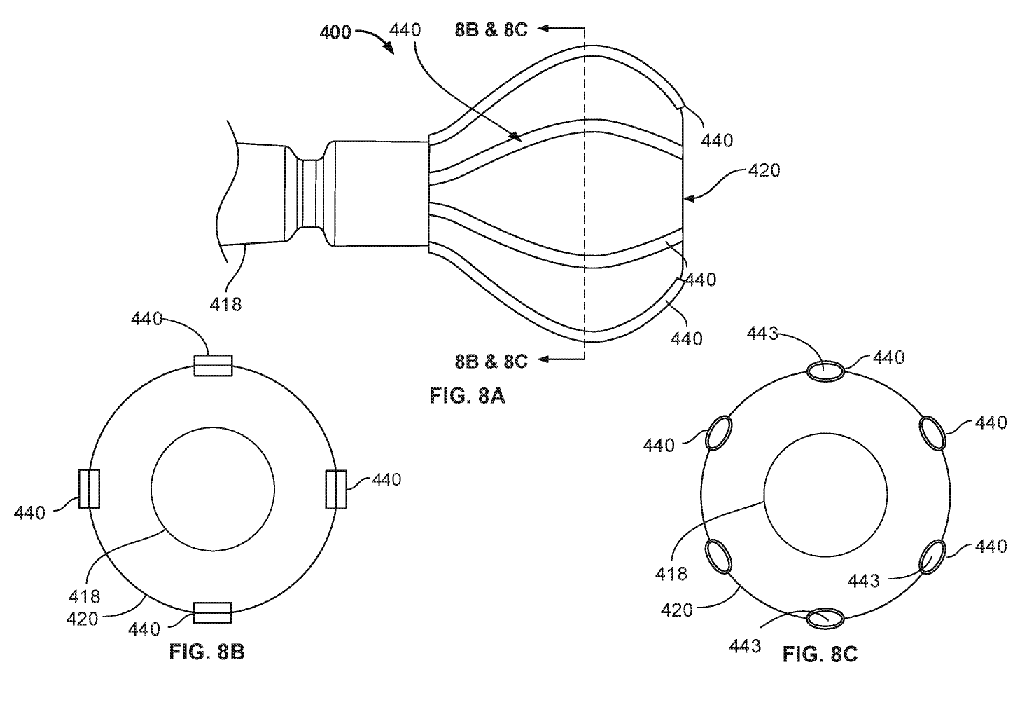

FIGS. 8A-8C are a side view and cross-sectional views of another example of a balloon catheter visualization device 400 provided herein. Certain embodiments of the device 400 provided herein can include one or more inflatable (also can be described as diametrically expandable) reinforcement features 440 (which may also be referred to as ribs, tubes, or strips) coupled to a weeping balloon 420. Each inflatable reinforcement features 440 can define a lumen 443 sized to receive an inflation media therein. As the inflatable reinforcement features 440 fills with the inflation media, the inflatable reinforcement features 440 diametrically expands. The lumen 443 can be configured to be inflated independently of the balloon 420. For example, the lumens 443 of the inflatable reinforcement features 440 can be separate lumens (or joined together as one lumen) within the system shaft 418 that are not a fluid connection with the lumen supplying inflation media to the balloon 420.

In some cases, the inflatable reinforcement features 440 can extend along a portion of the balloon 420 to provide additional strength (e.g., stiffness). For example, in some cases, the inflatable reinforcement features 440 extend fully or partially along the balloon 420 in a longitudinal direction. In some cases, the inflatable reinforcement features 440 can extend circumferentially along the balloon, or along any angled direction along the body of the balloon 420. In some cases, the plurality of inflatable reinforcement features 440 can be located circumferentially equidistant from one another. In some cases, the inflatable reinforcement feature can include a layer of material that covers a portion (e.g., proximal portion) of the balloon, as shown in FIG. 4, with a sealed distal edge such that an inflatable pocket can be formed between the balloon wall and the layer of material.

The inflatable reinforcement features 440 can allow a user to modify the strength characteristics of the device 400 during use. For example, the inflatable reinforcement features can 440 be inserted into a patient's body in a deflated state, as shown in FIG. 8B, to minimize the overall device profile during delivery. The inflatable reinforcement features 440 can be inflated as desired, as shown in FIG. 8C, to provide the balloon 420 with increased stiffness under certain use conditions, for example, when the device 400 is being pushed against a patient's anatomy. The inflatable reinforcement features 440, when inflated, can provide the balloon with increased stiffness to prevent undesirable buckling, bending, or balloon folding during device use.

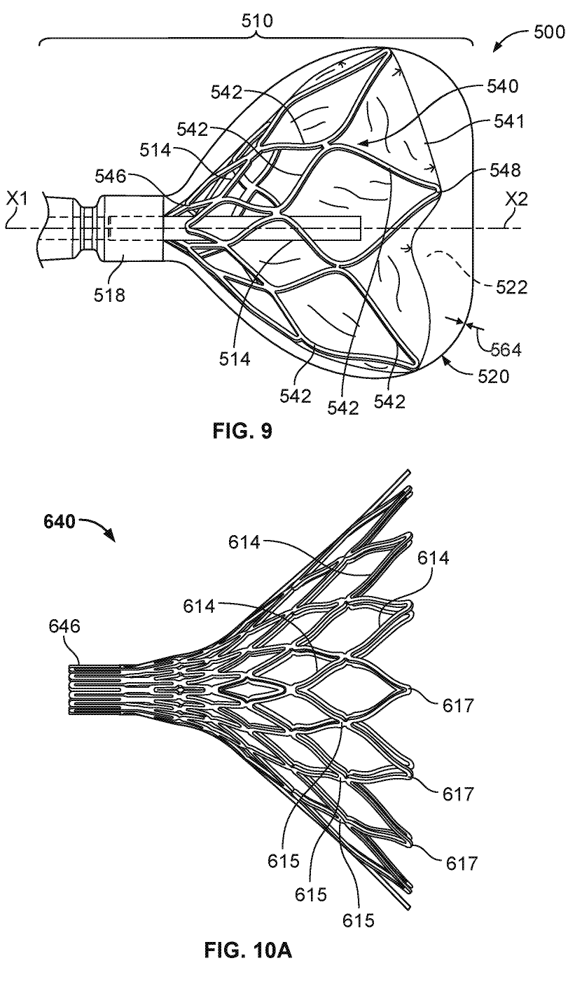

Referring to FIG. 9, certain embodiments of visualization balloon system 500 provided herein include a reinforcement feature, such as a reinforcement frame 540, disposed within an inflation cavity 522 of a weeping balloon 520. The visualization balloon system 500 can further include an outer shaft 518, and an inner member 514. The reinforcement frame 540 may be disposed within the inflation cavity 522 of the balloon 520. In some cases, the reinforcement frame 540 can be coupled to (e.g., bonded) to one or more portion of the balloon 520. For example, the reinforcement frame 540 may be coupled to an inner surface, or an outer surface of a balloon wall 564. In some cases, the reinforcement frame 540 can be embedded in the balloon wall 564.

In some cases, the reinforcement frame 540 can be a separate device that can be inserted into or, alternatively, withdrawn from the system 500. In particular, the reinforcement frame 540 may be inserted into or, alternatively, withdrawn from, the inflation cavity 522 of the balloon 520.

The reinforcement frame 540 of FIG. 9 includes the plurality of flexible struts 542 configured to expand and collapse together. Each strut 542 of the reinforcement frame 540 may be connected to at least two adjacent struts 542 such that all of the individual struts 542 of reinforcement frame 540 collapse or expand together. The reinforcement frame 540 can be configured include a collapsed configuration for providing a small profile for suitable for delivering it within the balloon 520 and an expanded configuration for providing structural support to the walls of the balloon 520. In some cases, the reinforcement frame 540 may be configured to provide structural support to the balloon 520 such that the central axis “X2” of the balloon 520 will remain aligned with the longitudinal axis “X1” of the shaft 518 when a force is applied to the balloon 520, in particular, when the balloon 520 is in the inflated (expanded) state, by providing additional structural support to the balloon 520. The balloon 520 may flex or bend only to the extent that its walls come in contact with the struts 542 of the reinforcement frame 540. Under such cases, the reinforcement frame 540 may provide the balloon with sufficient strength to withstand forces being applied to the balloon 520 to prevent undesirable buckling, bending, or balloon material folding.

Still referring to FIG. 9, the reinforcement frame 540 includes a body having a proximal end 546 and a distal end 548. Each strut 542 of the frame 540 extends from the proximal end 546 to the distal end 548. When the reinforcement frame 540 is in the expanded configuration, the diameter of frame 540 at the distal end 548 is larger than the diameter of the frame 540 at the proximal end 546. The body of the reinforcement frame 540 can form a flared cone shape, when in the expanded configuration. When in the expanded configuration, the reinforcement frame 540 can have a maximum diameter that ranges from about 2 mm to about 5 mm. When in the collapsed configuration, the reinforcement frame 540 can have a maximum diameter that ranges from about 1 mm to about 2 mm (e.g., from about 1.0 mm to about 1.25 mm, from about 1.25 mm to about 1.5 mm, from about 1.5 mm to about 2.0 mm).

Each strut 542 of the reinforcement frame 540 can include one of various cross-sectional shapes. The shape of the strut 542 can be, for example, rectangular, circular, oval, triangular, square-shaped, and the like. Each strut 542 can include a suitable cross-sectional width (e.g., width ranging from about 0.1 mm to about 2 mm) and cross-sectional area (e.g., area ranging from about 0.01 mm² to about 4 mm²) to provide adequate structural support to the balloon 520 for preventing undesirable buckling, bending, and/or balloon folding.

In some cases, the reinforcement frame 540 can be made from one of various metals including, but not limited to, stainless steel and nitinol. In some cases, the reinforcement frame 540 can be made of a polymer, for example, polycarbonate, polyether block amide, polyurethane, nylon, or the like.

Certain embodiments of the reinforcement frame 540 can be coupled to a covering 541. In some cases, the covering 541 can be coupled to the reinforcement frame 540 along portions of its interior surfaces, exterior surfaces, or both. The covering 541 can be configured to allow for uniform expansion of the frame 540 during inflation of the balloon 520, and/or facilitate uniform refolding of the balloon 520 during device system 500 withdrawal. In some cases, the covering 541 can be used to limit the expansion of the frame 540 within the balloon 520. The covering 541 of the reinforcement frame 540 within a balloon 520 may be used in some cases to prevent or reduce risk of the reinforcement frame 540 causing balloon damage, or tissue damage (e.g., dissection or tissue abrasion) through the balloon wall. The covering 541 can be made of a polymeric material, such as a polyurethane, polyamide (e.g., PEBAX), or a silicone. The covering 541 can be made using an extrusion or molding process. The covering 541 can be attached to the reinforcement frame 540 using an adhesive or a heat bonding application.

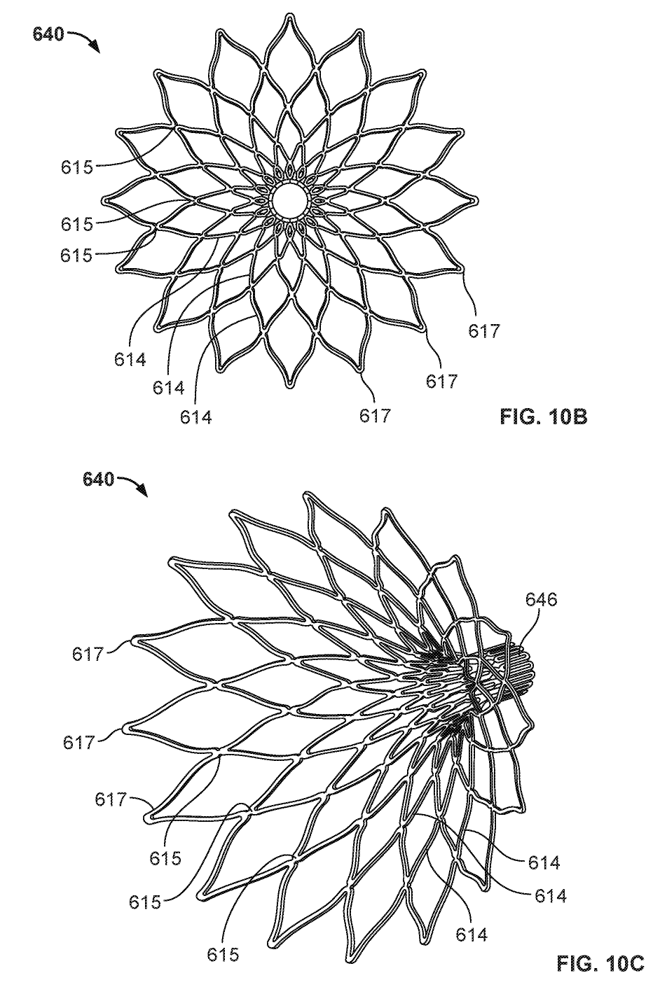

Referring to FIGS. 10A-10C, another example of a reinforcement feature in the form of a frame 640 including a plurality of interconnected struts 614, shown in an expanded state. The depicted reinforcement frame 640 has a petal formation in which a proximal end 646 of the structure is tubular in shape and gradually expands radially towards a distal end. The interconnected struts 614 are flexible such that the frame 640 can collapse into a tubular arrangement during device delivery.

The reinforcement frame 640 can include a plurality of interconnected struts 614 such that each individual strut 614 forms a petal-like shape. Each strut 614 can be bonded to another strut 614 at one or more overlapping, connecting regions 615 (e.g., at two, three, four, five, six, seven, eight, nine, ten, or more than ten connecting regions). Each strut 614 extends distally from the proximal end 646 to form a rounded distal tip 617 before extending back toward the proximal end 646.

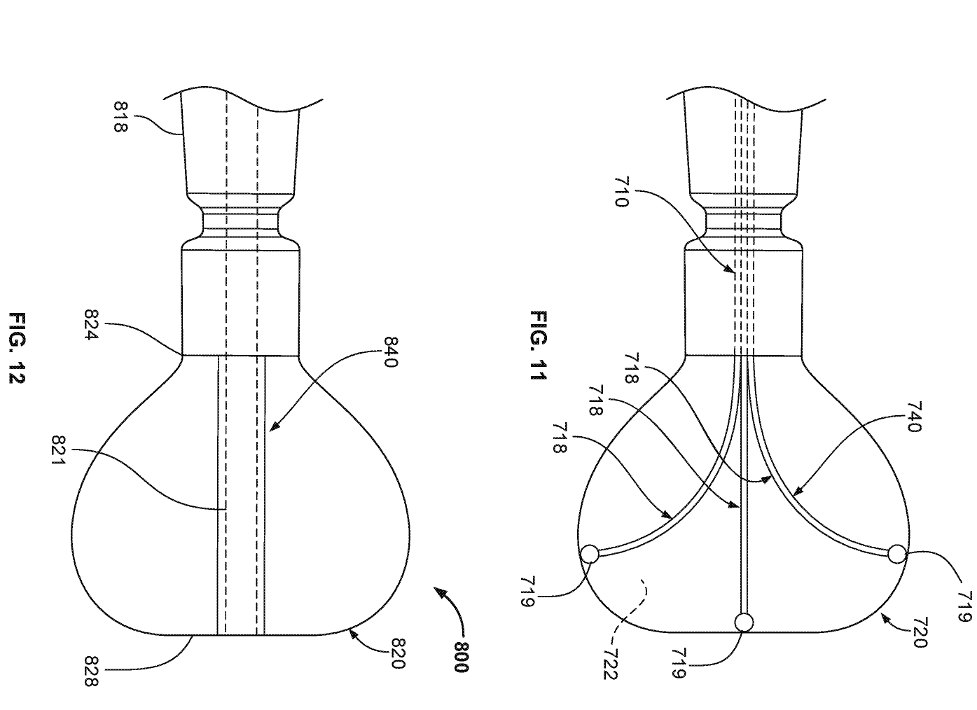

Referring to FIG. 11, a third example of a reinforcement feature 740 that includes three-pronged member 718 disposed within the balloon interior cavity 722. The depicted reinforcement feature 740 includes a proximal portion and a distal portion. The proximal portion includes a rod 710 and the distal portion includes three radially expandable prongs 718 configured to expand and contact different portions of a weeping balloon 720 within its interior cavity 722. The prongs 718 can provide structural support to the balloon 720 to prevent the balloon 720 from buckling, bending, and/or folding on itself. The reinforcement feature 740 can be a separate component or device that is inserted into and withdrawn from a balloon inner cavity.

The reinforcement feature 740 of FIG. 11 can include a metal (e.g., stainless steel, or nitinol), a polymer (e.g., polycarbonate, a polyether block amide, or polyimide), or a combination thereof. For example, in some cases, the pronged reinforcement feature 740 may include a flexible, shape memory material that can be easily collapsed and expanded upon shape memory activation.

Still referring to FIG. 11, each prong 718 of reinforcement feature 740 may optionally include balled tips 719 configured for minimizing perforation of the prongs tips through the wall of the balloon 720. In some cases, the balled tips 719 can include a radiopaque material configured for providing radiopaque markers during a medical procedure. The balled tips 719 can be made of a metal (e.g., nitinol, nickel, gold, silver, titanium, and alloys), polymer (e.g., PEBAX®, silicone, polyurethane), or combinations thereof (e.g., radiopaque metal particles within a polymer matrix).

Referring to FIG. 12, a fourth example of a rigid or semi-rigid inner reinforcement feature 840 disposed within the balloon 820. The inner reinforcement feature 840 can be coupled to an outer shaft 818 and to the distal end 828 of the balloon 820. The inner reinforcement feature 840 can extend through the entire balloon, from the proximal end 824 of the balloon 820 to the distal end 828 of the balloon 820. In some cases, the inner reinforcement feature can include various shapes, such as a tubular, elliptical, or ovate shape. In some cases, as shown, the inner reinforcement feature 840 may be concentric with a balloon lumen, for example, the inner reinforcement feature 840 forms a lumen 821 (which can also be described as a working channel) through the balloon 820. In some cases, the inner reinforcement feature 840 can be non-concentric with the balloon lumen. For example, the inner reinforcement feature 840 may extend, parallel to the balloon lumen, from the outer shaft 818 into an inflation cavity of the balloon 820

In some cases, the inner reinforcement feature 840 can define the lumen 821 configured for allowing other components or device to pass through. The lumen 821 can be configured to allow other components and devices to extend through the balloon 820 without damaging (e.g., perforating) the balloon 820. The inner reinforcement feature 840 can also provide additional mechanical strength (e.g., stiffness) to the balloon 820 to reduce or prevent bending, buckling, and material folding of the balloon 820 when a force is applied to the balloon 820.

The inner reinforcement feature 840 can be made of a material having higher stiffness properties than the balloon material. For example, the inner reinforcement feature 840 can include a semi-rigid polymer, or a composite material, such as soft polymer embedded with reinforcement elements, such metallic or ceramic fillers, braid, or longitudinal members (e.g., fibers or wires).

A number of embodiments of the reinforcement features for use with balloon catheter visualization devices and other medical devices, systems, and methods have been described. Nevertheless, it will be understood that various modifications may be made without departing from the spirit and scope of the subject matter described herein. Moreover, it should be understood that the features of one or more of the weeping balloon devices described herein can be combined with features from one or more other weeping balloon devices provided herein. That is, hybrid designs can be created by combining various features and such hybrid designs are fully within the scope of this disclosure. Accordingly, other embodiments are within the scope of the disclosure and the following claims.

We claim:

1. A balloon catheter visualization device comprising:
a handle;
an elongate shaft comprising a distal end and a proximal end, the proximal end coupled to the handle, the elongate shaft defining a lumen and a longitudinal axis that extend from the proximal end to the distal end; and
an asymmetrical-shaped balloon comprising a distal portion and a proximal portion, the proximal end coupled to the distal end of the elongate shaft, the proximal portion including one or more reinforcement features comprising a layer of material coupled to the balloon and forming an outer contour at the proximal portion of the balloon that is asymmetrical to an outer contour of the distal portion of the balloon.

2. The balloon catheter visualization device of claim 1, wherein the asymmetrical-shaped balloon comprises multiple reinforcement features that form a plurality of ribs.

3. The balloon catheter visualization device of claim 2, wherein the plurality of ribs are located circumferentially equidistant from one another.

4. The balloon catheter visualization device of claim 1, wherein the one or more reinforcement features comprises at least one diametrically expandable rib.

5. The balloon catheter visualization device of claim 4, wherein the at least one diametrically expandable rib defines a lumen sized to receive an inflation media for diametrically expanding the rib.

6. The balloon catheter visualization device of claim 4, wherein the at least one diametrically expandable rib is configured to be inflated independently of the asymmetrical-shaped balloon.

7. The balloon catheter visualization device of claim 1, wherein the balloon catheter visualization device includes up to six or more reinforcement features.

8. The balloon catheter visualization device of claim 1, wherein the balloon catheter visualization device includes one reinforcement feature comprising the layer of material uniformly disposed over the proximal portion of the asymmetrical-shaped balloon.

9. The balloon catheter visualization device of claim 1, wherein the proximal portion has an average wall thickness that is greater than an average wall thickness of the distal portion.

10. The balloon catheter visualization device of claim 1, wherein a thickness ratio of a balloon wall coupled to the reinforcement feature in comparison to the balloon wall alone is about 1:1, about 2:1, about 3:1, about 4:1, about 5:1, or greater than 5:1.

11. The balloon catheter visualization device of claim 1, wherein the one or more reinforcement features are configured to prevent material folding at the proximal portion of the asymmetrical-shaped balloon, when the distal portion of the asymmetrical-shaped balloon is subjected to compressional force.

12. A balloon catheter visualization device comprising:
a handle;
an elongate shaft comprising a distal end and a proximal end, the proximal end coupled to the handle, the elongate shaft defining a lumen and a longitudinal axis that extend from the proximal end to the distal end;
a balloon comprising a proximal end coupled to the distal end of the elongate shaft and a distal portion, the balloon including a wall that defines an inner inflation cavity; and
a reinforcement feature disposed within the inner inflation cavity, the reinforcement feature comprising a plurality of flexible struts, each of the struts being connected to at least two adjacent struts, the reinforcement feature configured to include a collapsed configuration for providing a small profile suitable for delivery and an expanded configuration for providing structural support to the balloon.

13. The balloon catheter visualization device of claim 12, wherein the reinforcement feature in the expanded configuration forms a frame having a flared cone shape.

14. The balloon catheter visualization device of claim 12, wherein the reinforcement feature is a separate component configured to be inserted into the balloon and expanded therein.

15. The balloon catheter visualization device of claim 12, wherein the reinforcement feature is coupled at one or more locations of the balloon.

16. The balloon catheter visualization device of claim 12, wherein the reinforcement feature is coupled to a polymeric covering.

17. The balloon catheter visualization device of claim 12, wherein the reinforcement feature includes a plurality of interconnected struts, each individual strut forming a petal-like shape when the reinforcement feature is in the expanded configuration.

18. The balloon catheter visualization device of claim 12, wherein the reinforcement feature comprises nitinol.

19. The balloon catheter visualization device of claim 12, wherein the plurality of flexible struts are interconnected such that the struts collapse and expand together.

20. A balloon catheter visualization device comprising:
a handle;
an elongate shaft comprising a distal end and a proximal end, the proximal end coupled to the handle, the elongate shaft defining a lumen and a longitudinal axis that extend from the proximal end to the distal end; and
a balloon comprising a proximal end and a distal end, the proximal end of the balloon being coupled to the elongate shaft and comprising a means for reinforcing the balloon to prevent balloon material folding at the proximal end of the balloon when force is applied to the distal end of the balloon.
